(19) United States
(12) Patent Application Publication
(10) Pub. No.: US 2024/0401203 A1
(43) Pub. Date: Dec. 5, 2024

(54) Yeast (S. cerevisiae) sheathing through electrochemical discharge of Cobalt air battery

(71) Applicant: University of Sharjah, Sharjah (AE)

(72) Inventors: Enas Taha Sayed, Sharjah (AE); Mohammad Ali Abdelkareem, Sharjah (AE); Hussain Alawadhi, Sharjah (AE); AbdulGhani Olabi, Sharjah (AE)

(21) Appl. No.: 18/328,419

(22) Filed: Jun. 2, 2023

(51) Int. Cl. C25D 13/02; C12N 1/16; C12N 1/20; C25D 13/12; H01M 4/66; H01M 4/90; H01M 12/08; H01M 50/411; H01M 50/497

(52) U.S. Cl. CPC C23C 18/1662; C23C 18/1641; C23C 18/34; H01M 4/38; H01M 4/381; H01M 4/382; H01M 4/663; H01M 4/926; H01M 12/08; H01M 50/426

(57) ABSTRACT

Novel method for the encapsulation of yeast cells through electrochemical discharge of a Cobalt air battery.

### TECHNICAL FIELD

The present invention relates to novel method for cell encapsulation of yeast cells using a facile single-step electrochemical method while generating electricity in a Co air metal battery.

### BACKGROUND

Cell Encapsulation with metal and/or polymer shells is a critical elongation of cellular functions. Encapsulating living cells plays a crucial role in the progression of cell surface engineering and biomedical applications such as tissue engineering, cell-based transplantation therapy, cell-based biosensors, and drug delivery [1, 2]. Moreover, encapsulating living cells could introduce an isolated and well-defined microenvironment mimicking the extracellular matrix of natural tissues to observe the modulation of cell function, the microenvironment interaction of the cell, food industries, and pharmaceutical applications [3, 4]. The hydrated polymers cross-linked hydrogels technique is considered a standard cell encapsulation process [5]. However, this technique has several limitations, such as short sustenance time, limited stability, and low permeability, limiting its practical application [6, 7]. Other methods including biothreading [8], assisted biojetting [9, 10], bio-electrospraying, and cell electrospinning [11, 12], use air pressure or electric field as a driving force. Until now, the layer-by-layer self-assembly technique is considered the most proper cell encapsulation strategy [13]. All these methods are complicated, time-consuming, and need a lot of effort and energy input.

Several cell and tissue engineering techniques, i.e., cell patterning, gene transfer, tissue growth, viral transduction, have been applied to nanoparticles [14-18]. The electrical, mechanical, energy, and optical properties changed according to the type of the nanoparticles. The living cell can internalize the nanoparticles through the cell membrane [19, 20]. For example, iron oxide nanoparticles with polycations encapsulated bacteria Alcanivorax borkumensis with a magnetic nanocoating layer. The encapsulated bacteria cells fabricating live bacteria cells with magnetic properties [21].

Probiotics are good microorganisms that live in our bodies, keeping us healthy. According to The Food and Agriculture Association of the United Nations and World Health Organization (WHO), “probiotics are live microorganisms which when administered in adequate amounts confer a health benefit on the host.” The main types of probiotics are Saccharomyces, Bifidobacterium, Lactobacillus, and Bacillus species. The probiotics increase the number of useful microorganisms, inhibiting the growth of harmful bacteria, enhancing the diversity of the microorganism community, and assisting the body's defense system [22]. Probiotics have been proved to be useful treat diseases in the skin, oral cavity, vagina, etc. [23-26]. The viability and activity of probiotics are affected by the organ/tissue environment factors such as osmotic pressure, acids, ions concentrations, bile, oxidative stress, and nutrient consumption [27-29]. Encapsulation or immobilization of probiotics using microencapsulation or electrospinning technique was introduced to conquer the above issues resulting in the application of probiotics in various areas.

S. cerevisiae is a model organism for different research areas and is widely used in various industrial applications [30]. S. cerevisiae is considered very adaptive microorganisms, and they can survive under both anaerobic and aerobic conditions [31]. Yeast cell has an inner membrane surrounding its intracellular components and an external cell wall.

Therefore, there is a need for a new environmentally friendly simple method that can used at room temperature with no energy consumption, utilizing a variety of microorganism for medical and industrial applications.

### SUMMARY

In one embodiment of the present disclosure, disclosed herein is an electrochemical system for encapsulating a probiotic cell with a metallic surface by electrodeposition, the system comprising a two-electrode metal-air battery comprising a cathode, a separator, a carbon cloth, an anode, and an anolyte solution, wherein:
(a) the cathode comprises a Pt/C material;
(b) the separator comprises an ion-conductive membrane;
(c) the anode comprises a carbon cloth modified with a metal nanoshell structure;
(d) the anolyte comprises a probiotic cell.

In one aspect of the present disclosure, disclosed herein is a method for encapsulating a probiotic cell utilizing the electrochemical system described above, wherein the electrodeposition comprises the step of electrochemically discharging a metal-air battery to form a metallic sheath on the surface of a probiotic cell, wherein the metallic sheath comprises a metal.

In a most preferred aspect of the present disclosure, the metal is cobalt.

In one aspect of the present disclosure, the metal utilized can be cobalt, zinc, aluminium, magnesium, lithium, iron, zirconium, tungsten, and vanadium.

In a preferred aspect of the present disclosure, the metal air battery utilizes cobalt.

In one aspect of the present disclosure, the metal air battery may utilize cobalt, zinc, aluminium, magnesium, lithium, iron, zirconium, tungsten, or vanadium.

In a preferred aspect of the present disclosure, the metallic sheath comprises cobalt.

In a preferred aspect of the present disclosure, the ion-conductive membrane is a polymer or powder resin membrane.

In a most preferred aspect of the present disclosure, the membrane is a Nafion® membrane.

In one aspect of the present disclosure, the metallic sheath has magnetic properties.

In a preferred aspect of the present disclosure, the probiotic cell is a yeast cell.

In a most preferred aspect of the present disclosure, the yeast cell is a Saccharomyces cerevisiae cell.

In a preferred aspect of the present disclosure, the probiotic is a microorganism that may include Saccharomyces, Bifidobacterium, Lactobacillus, and Bacillus.

In one aspect of the present disclosure, the system operates at room temperature.

In a preferred aspect of the present disclosure, no current is consumed.

In a most preferred aspect of the present disclosure, current is produced.

In a preferred aspect of the present disclosure, the current produced by the battery during discharge has a density of approximately 3 A/m² at 0.3 V.

In one aspect of the present disclosure, the probiotic encapsulated cell is viable and capable of reproducing.

In a preferred aspect of the present disclosure, the magnetic properties of the metallic sheath encapsulating the probiotic cell may be utilized in a fuel cell.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 provides a schematic diagram of the cell.

FIG. 2 provides the SEM images of the plain carbon cloth (FIG. 2a) and carbon cloth with the metallic layer (Cobalt nanosheet structure (FIG. 2b)). 2 (a) Plain carbon cloth (CC) 2 (b) Carbon cloth (CC) coated with Co nanosheet structure

FIG. 3 provides EDX analysis of the Co on the surface of the carbon cloth (CC). The EDX analysis shows that the prepared material is composed mainly of the Co metal with a small amount of surface oxygen. At the same time, the appeared carbon is related underneath carbon fibers.

FIG. 4 provides (a) i-V and i-P and (b) current discharge of the cobalt air battery using Co/CC anode.

FIG. 5 provides SEM images of the anode after the current discharge in metal air battery (a and b) low resolution, and (c and d) high resolution. The whole surface is covered by the yeast cells (FIG. 5 a and b), and the yeast cells themselves are sheathed (FIG. 5c), and this sheath is porous and rough (FIG. 5d). It was clear from the SEM images at the end of the experiments that the surface Co disappeared from the surface of the carbon cloth fibers while yeast cells were sheathed with a porous metallic layer. The EDX analysis of the yeast cells sheathed with Cobalt is shown in FIG. 6. The EDX analysis clearly shows that the shell is composed of more than 20 wt % of Cobalt in the whole surface.

FIG. 6 provides EDX analysis of the yeast cells sheathed with Cobalt at the end of the current discharge.

FIG. 7 provides a schematic of the cell during discharged initial (left), and after some time (right). The cell then worked under closed-circuit conditions, as shown in FIG. 7. The cell showed a current discharge of 3 A/m² at 0.3 V. After two hours of cell operation, the cell was opened, and the morphology of the anode and yeast cells in the anolyte was investigated as shown in FIG. 5.

FIG. 8 provides (a) i-V and i-P using Co/CC electrode without glucose, (b) current discharge using Co/CC electrode without glucose.

FIG. 9 (a and b) SEM images of the electrode at the end of the experiment, and (c) areal EDX elemental analysis of spectrum 13 shown in FIG. 8(b).

FIG. 10 provides SEM images showing the division of the yeast cells over Cobalt modified carbon cloth. Several points were taken to confirm that the sheathing did not affect the division of the yeast cells, as shown in FIG. 10. As being clear from the figures that the yeast cells are in the reproduction stage (divide) in several parts, such as clear in the high magnification of the point (spectrum 13 and high magnification images), indicating that these yeast cells sheathed and this sheathing did not affect their reproduction.

### DEFINITIONS

As used in this description and the accompanying claims, the following terms shall have the meanings indicated, unless the context otherwise requires:

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “scaffold” in accordance with the present invention, includes scaffold, body, block, chip, substrate, matrix, or segment.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

### DETAILED DESCRIPTION

A study was conducted to identify a novel method for the sheathing of yeast cells utilizing a facile electrochemical process, i.e. electrochemical discharge of Cobalt air battery.

Yeast cells are considered an efficient bio-system for the synthesis of metal nanostructures. S. cerevisiae is a model organism for different research areas and is widely used in various industrial applications [32]. S. cerevisiae is a very adaptive microorganism, and it can survive under anaerobic and aerobic conditions [33]. Yeast cells have an inner membrane surrounding their intracellular components and an external cell wall.

It has been reported that sheathing can form on the surface of the yeast cell through large complex steps similar to the case of the silicification of yeast cells [34]. Such a shell would increase yeast cells' long-term viability by stabilizing cellular membranes under physicochemical pressure, protecting the cellular structure from hydration, and improving the cell membrane's mechanical strength, chemical stability, and electrochemical properties [34].

In this study, a thin layer of cobalt nanostructure was synthesized on a carbon cloth surface (Co/CC) by a simple electrophoretic process. The synthesized Co/CC was used as an anode of a Cobalt air battery containing yeast cells in the anolyte solution. During the current discharge of the cobalt air battery, yeast cells are sheathed with a metallic layer of cobalt. The yeast cells go into a division process, indicating that the sheathing process does not negatively affect the yeast cells. The proposed method is simple, operated at room temperature, is an energy-producing method, and can be applied to various types of similar microorganisms. The results of the current work will open the door for a large number of applications.

In a first aspect, disclosed herein is a method for enclosing yeast cells with a metallic surface through a facile electrochemical process, i.e., electrochemical discharge of Cobalt air battery operated at room temperature.

In a second aspect, the novel method discloses a unique and simple sheathing method that operates at room temperature and an energy-producing method.

In a third aspect, the yeast cells encapsulated by the method of the present invention have a Co shell that has magnetic properties.

In one embodiment of the present disclosure, disclosed herein is an electrochemical system for encapsulating a probiotic cell with a metallic surface by electrodeposition, the system comprising a two-electrode metal-air battery comprising a cathode, a separator, a carbon cloth, an anode, and an anolyte solution, wherein:
(e) the cathode comprises a Pt/C material;
(f) the separator comprises an ion-conductive membrane;
(g) the anode comprises a carbon cloth modified with a metal nanoshell structure;
(h) the anolyte comprises a probiotic cell.

In one aspect of the present disclosure, disclosed herein is a method for encapsulating a probiotic cell utilizing the electrochemical system described above, wherein the electrodeposition comprises the step of electrochemically discharging a metal-air battery to form a metallic sheath on the surface of a probiotic cell, wherein the metallic sheath comprises a metal.

In a most preferred aspect of the present disclosure, the metal is cobalt.

In one aspect of the present disclosure, the metal utilized can be cobalt, zinc, aluminium, magnesium, lithium, iron, zirconium, tungsten, and vanadium.

In a preferred aspect of the present disclosure, the metal air battery utilizes cobalt.

In one aspect of the present disclosure, the metal air battery may utilize cobalt, zinc, aluminium, magnesium, lithium, iron, zirconium, tungsten, or vanadium.

In a preferred aspect of the present disclosure, the metallic sheath may comprise a metal including cobalt, zinc, aluminium, magnesium, lithium, iron, zirconium, tungsten, or vanadium.

In one aspect of the present disclosure, the metallic sheath comprises a metal

In a preferred aspect of the present disclosure, the membrane is an ion-conductive membrane.

In a preferred aspect of the present disclosure, the membrane is an proton-conductive membrane.

In a preferred aspect of the present disclosure, the ion-conductive membrane is a polymer or powder resin membrane.

In a most preferred aspect of the present disclosure, the membrane is a Nafion® membrane.

In a preferred aspect of the present disclosure, the ion-conductive membrane is selected from the group consisting of Nafion®, and NEXAR™.

In one aspect of the present disclosure, the metallic sheath has magnetic properties.

In a preferred aspect of the present disclosure, the probiotic cell is a yeast cell.

In a most preferred aspect of the present disclosure, the yeast cell is a Saccharomyces cerevisiae cell.

In a preferred aspect of the present disclosure, the probiotic is a microorganism that may include Saccharomyces, Bifidobacterium, Lactobacillus, and Bacillus.

In one aspect of the present disclosure, the method consists of a single-step.

In a preferred aspect of the present disclosure, no current is consumed.

In a most preferred aspect of the present disclosure, current is produced.

In a preferred aspect of the present disclosure, the current produced has a density of approximately 3 A/m² at 0.3 V.

In one aspect of the present disclosure, the probiotic encapsulated cell is viable and capable of reproducing.

In a preferred aspect of the present disclosure, the magnetic properties of the metallic sheath encapsulating the probiotic cell may be utilized in a fuel cell.

In one aspect of the present disclosure, the anode, membrane, and cathode are fixed between two stainless steel current collectors having a thickness of approximately 1.0 mm.

In one aspect of the present disclosure, the buffer solution of the anolyte has a pH of approximately 7.0.

In one aspect of the present disclosure, the encapsulated yeast cells can be used in medical applications.

### EXPERIMENTAL EXAMPLES

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

### Example 1

Yeast Cells Encapsulation with Metallic Cobalt

Experiments have been conducted using a conducted using a two-electrode cell structure (Cobalt metal-air battery structure) composed of Pt/C on the surface of carbon cloth as the cell's cathode, Nafion membrane as a separator, and carbon cloth modified with the Co nanoshell structure at the anode side. S. cerevisiae yeast cells were used in the anolyte of the cell using buffer solution at pH 7.

FIG. 2 shows the SEM images of the CC before (FIG. 2a) and after electrophoretic deposition of Co (FIG. 2b). This figure shows that the plain CC (FIG. 2a) comprises smooth carbon fibers of around 6 μm average diameter. These fibers were covered with nano-sheet structures of Co after the electrophoretic process (FIG. 2b). Co/CC's elemental analysis demonstrated that the surface is mainly metallic cobalt, with small amounts of carbon-related to the underneath carbon fibers and small amounts of surface oxygen (FIG. 2c).

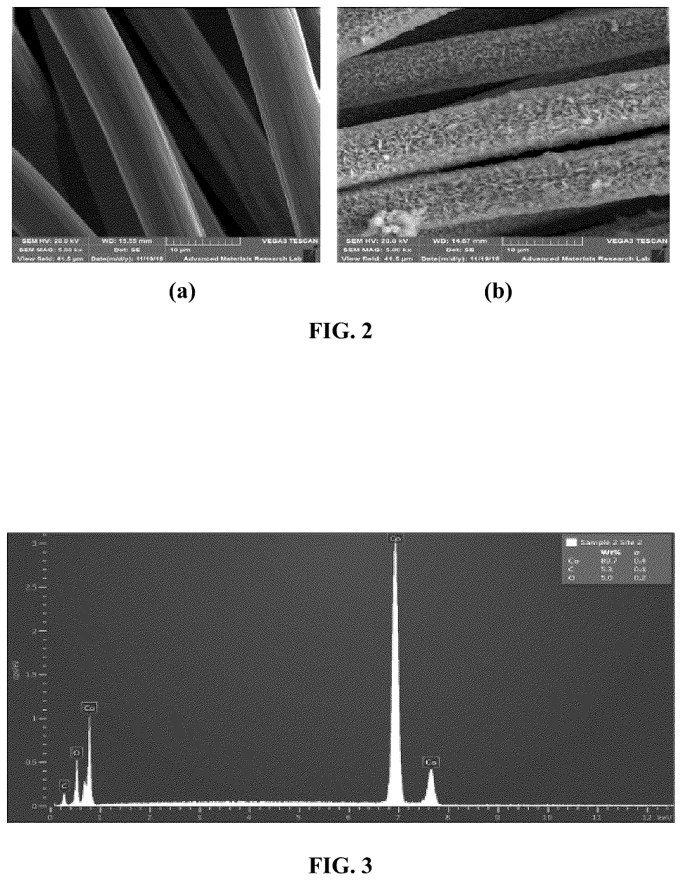

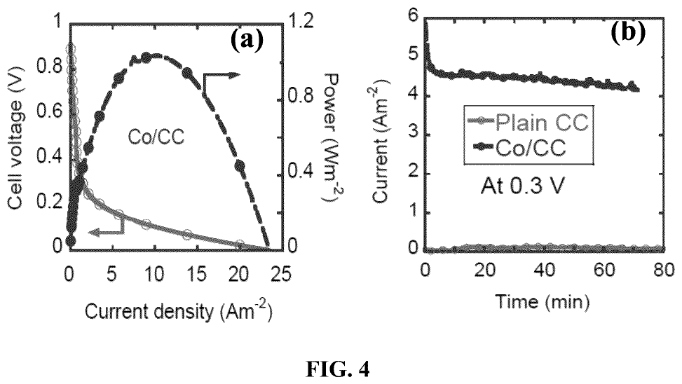

Cobalt air battery using Co/CC anode was operated from open-circuit voltage (OCV) to zero voltage at a voltage scan rate of 1 mV/s. The cell is successfully operated, producing a considerable power output of 1W/m². The current obtained in FIG. 3a represents an instantaneous operating condition of the cobalt air battery; the current discharge of the cell using plain CC and Co/CC anodes at 0.3 V was investigated for a longer time, FIG. 4b. As being clear from the figure, the cell successfully discharges a stable current density of 4.5 Am⁻² in the case of Co/CC anode compared to a negligible current discharge in the plain CC anode. The current discharge in the case of the Co/CC would be related to the dissolution of the Co metal (Co/CC) as follow:

Co→Co⁺²+2e⁻ (1)

While in the case of using plain CC, a negligible current is produced due to the usage of the metal-free anode, i.e., plain CC.

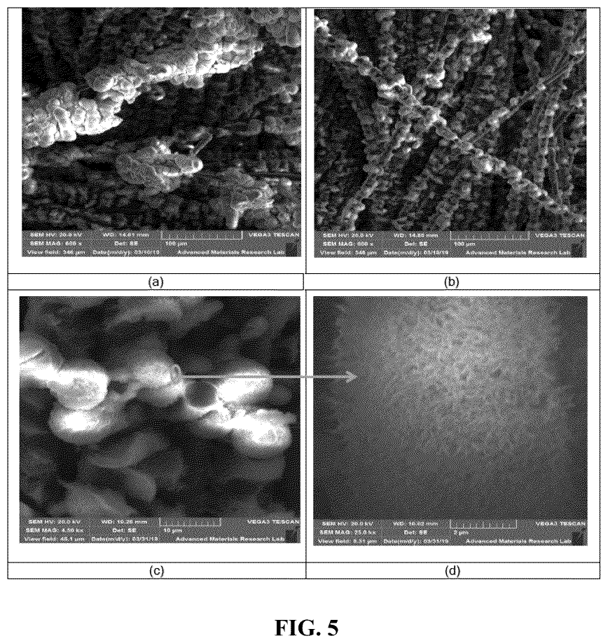

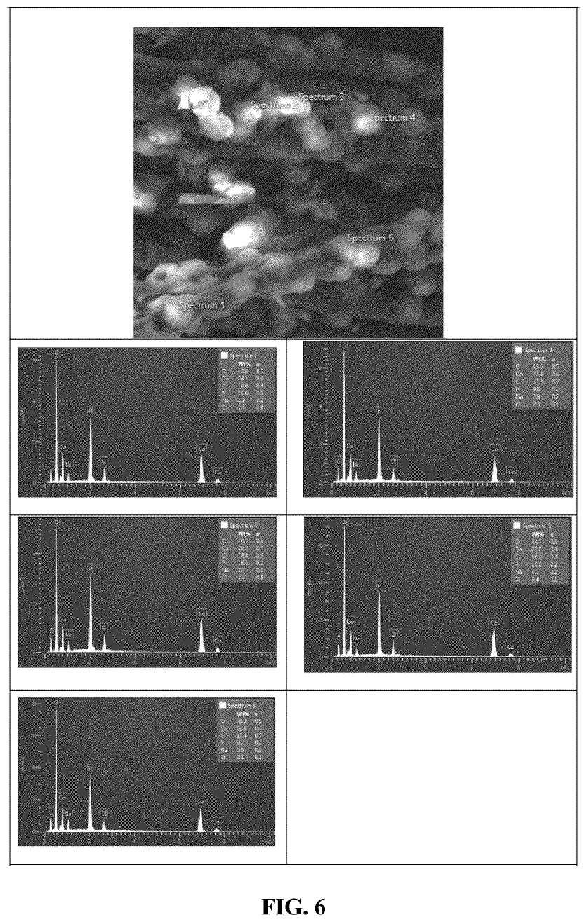

It is crucial to investigate the Co/CC anodes' surface morphology after completing electrochemical measurements (FIG. 5). It was clear from the SEM images that the initial sheet structure of the Co disappeared from the surface of the carbon cloth fibers, while yeast cells were sheathed with a porous metallic layer, FIG. 5a. At higher magnifications (FIGS. 5b and 5c), these yeast cells show a shiny metallic surface like pearls. Further magnification revealed that the yeast cells are sheathed with a rough, porous layer (insets of FIGS. 5b and c). The composition of this layer was determined using EDX analysis as shown in FIG. 6. Several points were selected. The surface has almost the exact composition of C, Co, O, and P. Also, it was clear from the EDX analysis that the sheath has a similar composition to that of the cell's outer layer, indicating that this sheath is a part of the cell. The collected yeast cells in the solution have the same characteristics as those on the surface of the carbon cloth. Furthermore, these yeast cells demonstrated a magnetic property as they were attracted and collected using a small magnet.

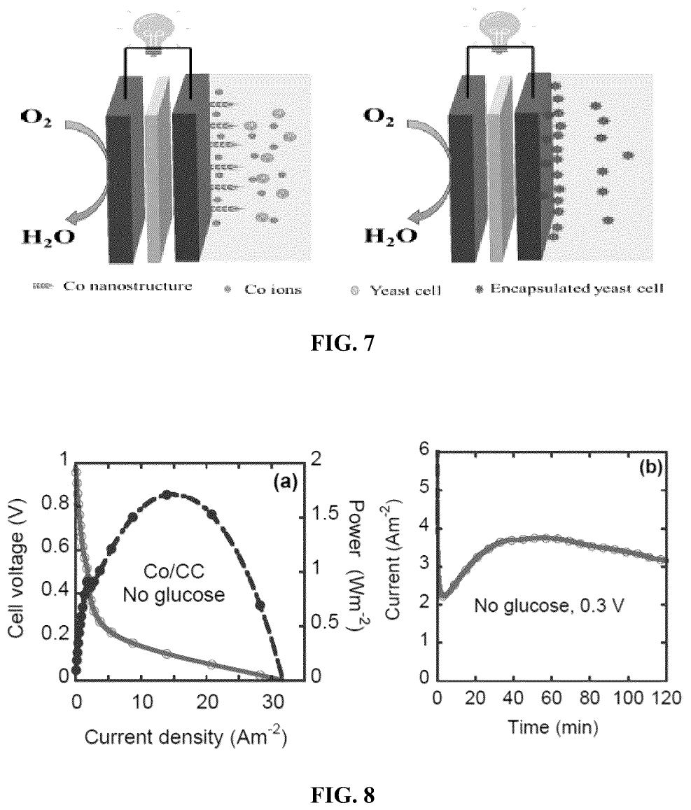

The mechanism of the sheathing process can be schematically presented in FIG. 7. During the current discharge of the Cobalt air battery, cobalt oxidation, i.e., dissolution, occurs on the anode, as expressed in equation 1. The concentration of the cobalt ions in the anolyte solution increased with increasing the discharging time, FIG. 5b. The cobalt ions produced in the anolyte solution could be reduced inside the yeast cells and exerted on its surface as follow:

Metal nanostructures can be synthesized biologically through the intracellular or extracellular mechanism. Microorganisms commonly excrete extra polysaccharide materials such as lipopolysaccharide, glycoprotein, etc., that possess anionic functional groups. Such anionic function groups attract cations from aqueous solutions (cobalt ions in this case). Bio-reduction involves the chemical reduction of metal ions to zero-valent or magnetic metal nanostructures via various reducing agents in microbial cell-like alkaloids, amides, amines, proteins, carbonyl groups, etc. In Intracellular synthesis, the metal ions are transferred to the inside of the microbial cell, then posterior reduction of metal ions into metallic nanoparticles, and lastly, dismissal of synthesized nanoparticles outside the microbial cell. Whereas extracellular synthesis comprises the bio-sorption of metal ions at the surface of the microbial cell, then posterior reduction of metal ions to metallic nanoparticles, and this approach has a wide range of applications.

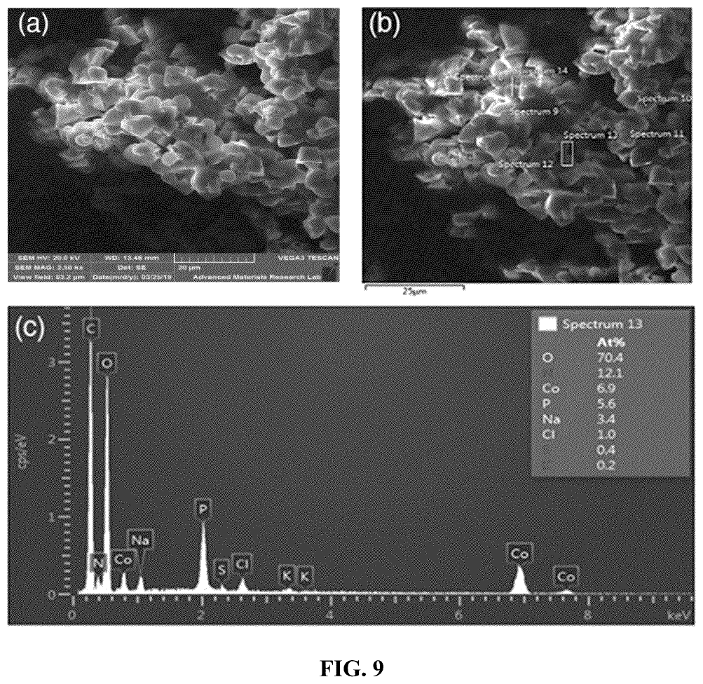

To examine the influence of glucose (food for the yeast cells) on the current discharge of the Co air battery and the sheathing process, the experiments were repeated without adding any glucose into the anolyte. The electrochemical measurements were carried out, followed by a surface morphology examination at the end of the measurements. The instantaneous current generation is shown in FIG. 8a, and the current generation at 0.3 V is shown in FIG. 8b. While the surface morphology of the anode is shown in FIGS. 9a and 9b, and the elemental analysis is shown in FIG. 9c. It is clear from FIGS. 8a and 8b that the Co/CC air battery generates current, indicating that the main current is coming from the electrooxidation of the cobalt metal and no role of the glucose in the current generation. The SEM images (FIGS. 9a and 9b) reveal that the yeast cells are active and asexually reproduced by budding. The areal EDX analysis indicates the presence of relatively high Co concentrations during the reproduction process, as seen in the EDX mapping of FIG. 9c.

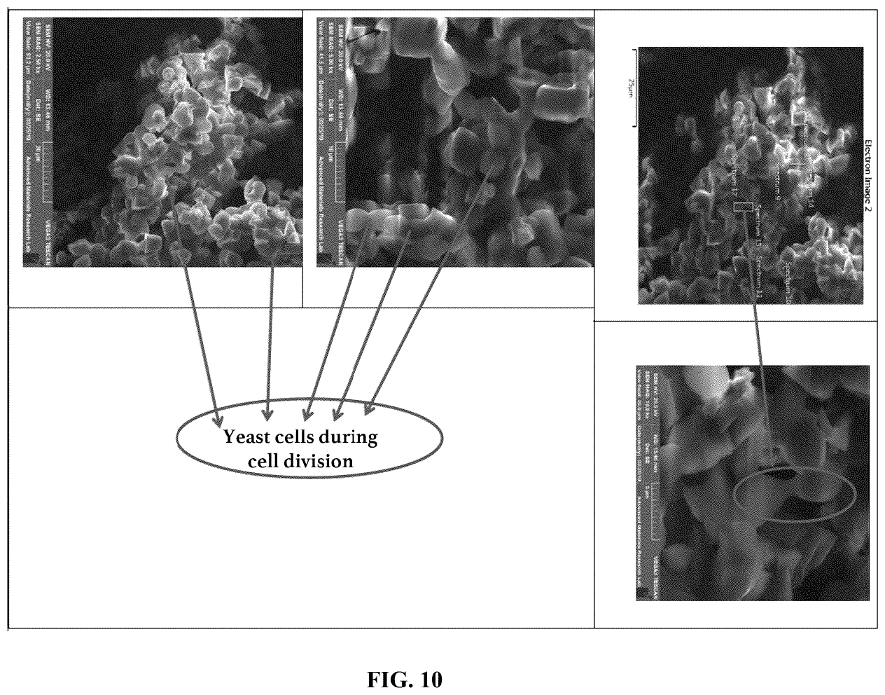

Several points were taken to confirm that the sheathing did not affect the division of the yeast cells, as shown in FIG. 10. As being clear from the figures that the yeast cells are in the reproduction stage (divide) in several parts, indicating that these yeast cells are sheathed, and this sheathing did not affect their reproduction.

Yeast cells are typically encapsulated by Co shell that increases the long term viability of yeast cells through stabilization of cellular membranes under physicochemical pressure as well as protecting the cellular structure from hydration, improve the mechanical strength and chemical stability of the native cell membrane similar to those obtained in case of biomimetic silicification (that is complex and done in several steps) of yeast cells [35].

The metal shell can be further functionalized easily compared to complicated chemical and biological processes done in case of the absence of the metal shell [36-39].

### Materials and Methods

### Electrophoretic Deposition of Cobalt on Carbon Cloth

Carbon cloth (CC) of 3×5.5 cm² “EC-20-10, Electro Chem, Inc.” was immersed in acetone for 15 min, then in deionized water for 30 min, and finally dried at 60° C. in an oven in static air. Co was deposited onto the CC surface using a two-electrode cell structure of graphite rode as anode and CC as the cathode. The electrodeposition was conducted using 0.16 M of cobalt acetate under an external voltage of 10 V for 10 min. After washing, the prepared Co over carbon cloth (Co/CC) was dried in an oven at 50° C. overnight.

### Cobalt Air Battery

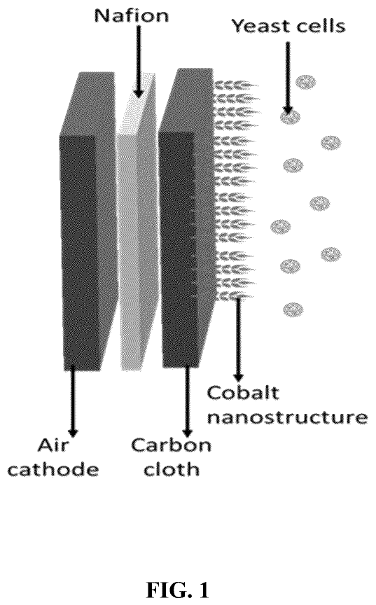

Experiments have been conducted using a two-electrode cell structure (Cobalt metal-air battery structure), as shown in FIG. 1. The cell is typically composed of Pt/C (0.5 mgcm⁻²)/C of 3×5.5 cm² “EC-20-10, ElectroChem, Inc.” was used as the cell's cathode, Nafion membrane as a separator, and carbon cloth modified with the Co nano-shell structure at the anode side. The yeast (S. cerevisiae) cells were included in the anolyte (buffer solution at pH 7). CC with and without Co layer was used as the anode. The anode, membrane, and cathode were fixed between two stainless steel current collectors “1.0 mm thickness”.

### Anolyte Preparation

0.13 g of yeast “Saccharomyces cerevisiae-Baker's yeast, (S.I. Lesa ffre), Marcq-en-Baroeul, France”, 0.5 g of glucose (Sigma Aldrich), 2 g of peptone (Sigma Aldrich), and 1 g of yeast extract (Sigma Aldrich) were added to distilled water “100 ml”; and then cultivated “for 16 h at 30° C.”. The anolyte “84 ml” was prepared by adding 10 ml of the prepared media to 74 ml of phosphate buffer (50 mM) to adjust the pH to 7.

### Electrochemical Performance

The two ends of the cell were connected to VSP 300 “a Bio-logic electrochemical station that used for performing the various electrochemical measurements, i.e., linear sweep voltammetry, chronoamperometry, and electrochemical impedance spectroscopy. Under closed-circuit conditions, the current output was determined while decreasing the cell voltage at 1 mV/s until it reached a zero voltage. The current density discharge was then measured at 0.3 V.

### Characterization

The CC's surface morphology and elemental composition with and without the Co layer before and after the electrochemical measurements were examined using a Scanning Electron Microscope with EDX (Tescan VEGAa XMU). After the electrochemical measurements, the anode was removed from the cell and kept drying in an oven at 50° C. for 3 hours before examination [40].

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

### REFERENCES

[1] J.-Z. Wang, Z.-Q. Ding, F. Zhang, W.-B. Ye, Recent development in cell encapsulations and their therapeutic applications, Materials Science and Engineering: C, 77 (2017) 1247-1260.
[2] B. Wang, P. Liu, Z. Liu, H. Pan, X. Xu, R. Tang, Biomimetic construction of cellular shell by adjusting the interfacial energy, Biotechnology and bioengineering, 111 (2014) 386-395.
[3] A. C. Wan, J. Y. Ying, Nanomaterials for in situ cell delivery and tissue regeneration, Advanced drug delivery reviews, 62 (2010) 731-740.
[4] C. R. Correia, R. P. Pirraco, M. T. Cerqueira, A. P. Marques, R. L. Reis, J. F. Mano, Semipermeable capsules wrapping a multifunctional and self-regulated co-culture microenvironment for osteogenic differentiation, Scientific reports, 6 (2016) 1-12.
[5] L. Gasperini, J. F. Mano, R. L. Reis, Natural polymers for the microencapsulation of cells, Journal of the royal society Interface, 11 (2014) 20140817.
[6] B. V. Slaughter, S. S. Khurshid, O. Z. Fisher, A. Khademhosseini, N. A. Peppas, Hydrogels in regenerative medicine, Advanced materials, 21 (2009) 3307-3329.
[7] R. R. Costa, C. A. Custódio, F. J. Arias, J. C. Rodríguez-Cabello, J. F. Mano, Layer-by-layer assembly of chitosan and recombinant biopolymers into biomimetic coatings with multiple stimuli-responsive properties, Small, 7 (2011) 2640-2649.
[8] Y. Choi, B. Phan, M. Tanaka, J. Hong, J. Choi, Methods and Applications of Biomolecular Surface Coatings on Individual Cells, ACS Applied Bio Materials, 3 (2020) 6556-6570.
[9] S. Arumuganathar, N. Suter, P. Walzel, S. N. Jayasinghe, Aerodynamically assisted jetting and threading for processing concentrated suspensions containing advanced structural, functional and biological materials, Biotechnology Journal: Healthcare Nutrition Technology, 4 (2009) 64-72.
[10] S. N. Jayasinghe, Biojets in regenerative biology & medicine, Materials Today, 14 (2011) 202-211.
[11] Â. Semitela, G. Ramalho, C. Sousa, A. Mendes, P. Marques, A. Completo, Bio-electrospraying assessment towards in situ chondrocyte-laden electrospun scaffold fabrication, Authorea Preprints, (2020).
[12] J. M. Ameer, R. Pai, R. Komeri, V. Damodaran, P. A. Kumar, N. Kasoju, Combinatorial approaches in electrospinning, electrospraying, and 3D printing for biomedical applications, in: Biomedical Applications of Electrospinning and Electrospraying, Elsevier, 2021, pp. 355-373. 10
[13] T. Liu, Y. Wang, W. Zhong, B. Li, K. Mequanint, G. Luo, M. Xing, Biomedical Applications of Layer-by-Layer Self-Assembly for Cell Encapsulation: Current Status and Future Perspectives, Advanced healthcare materials, 8 (2019) 1800939.
[14] K. Ino, A. Ito, H. Honda, Cell patterning using magnetite nanoparticles and magnetic force, Biotechnology and Bioengineering, 97 (2007) 1309-1317.
[15] J. Shen, J. Kim, S. Y. Tzeng, K. Ding, Z. Hafiz, D. Long, J. Wang, J. J. Green, P. A. Campochiaro, Suprachoroidal gene transfer with nonviral nanoparticles, Science advances, 6 (2020) eaba1606.
[16] R. Eivazzadeh-Keihan, E. Bahojb Noruzi, K. Khanmohammadi Chenab, A. Jafari, F. Radinekiyan, S. M. Hashemi, F. Ahmadpour, A. Behboudi, J. Mosafer, A. Mokhtarzadeh, Metal-based nanoparticles for bone tissue engineering, Journal of Tissue Engineering and Regenerative Medicine, 14 (2020) 1687-1714.
[17] N. Tran, T. J. Webster, Magnetic nanoparticles: biomedical applications and challenges, Journal of Materials Chemistry, 20 (2010) 8760-8767.
[18] B. Kurena, A. Vežāne, D. Skrastiņa, O. Trofimova, A. Zajakina, Magnetic nanoparticles for efficient cell transduction with Semliki Forest virus, Journal of virological methods, 245 (2017) 28-34.
[19] P. Parasuraman, A. P. Antony, A. Sharan, B. Siddhardha, K. Kasinathan, N. A. Bahkali, T. M. Dawoud, A. Syed, Antimicrobial photodynamic activity of toluidine blue encapsulated in mesoporous silica nanoparticles against Pseudomonas aeruginosa and Staphylococcus aureus, Biofouling, 35 (2019) 89-103.
[20] W. Zhang, C. Xu, G.-Q. Yin, X.-E. Zhang, Q. Wang, F. Li, Encapsulation of inorganic nanomaterials inside virus-based nanoparticles for bioimaging, Nanotheranostics, 1 (2017) 358.
[21] S. A. Konnova, Y. M. Lvov, R. F. Fakhrullin, Nanoshell assembly for magnet-responsive oil-degrading bacteria, Langmuir, 32 (2016) 12552-12558.
[22] C. Dunne, Adaptation of bacteria to the intestinal niche: probiotics and gut disorder, Inflammatory bowel diseases, 7 (2001) 136-145.
[23] S.-K. Han, Y.-J. Shin, D.-Y. Lee, K. M. Kim, S.-J. Yang, D. S. Kim, J.-W. Choi, S. Lee, D.-H. Kim, Lactobacillus rhamnosus HDB1258 modulates gut microbiota-mediated immune response in mice with or without lipopolysaccharide-induced systemic inflammation, BMC microbiology, 21 (2021) 1-15.
[24] H. J. Jang, J. H. Kim, N.-K. Lee, H.-D. Paik, Inhibitory effects of Lactobacillus brevis KU15153 against Streptococcus mutans KCTC 5316 causing dental caries, Microbial Pathogenesis, 157 (2021) 104938.
[25] P. Chandrashekhar, F. Minooei, W. Arreguin, M. Masigol, J. M. Steinbach-Rankins, Perspectives on Existing and Novel Alternative Intravaginal Probiotic Delivery Methods in the Context of Bacterial Vaginosis Infection, The AAPS Journal, 23 (2021) 1-16.
[26] C. Callewaert, N. Knödlseder, A. Karoglan, M. Güell, B. Paetzold, Skin microbiome transplantation and manipulation: Current state of the art, Computational and Structural Biotechnology Journal, (2021).
[27] L. Cassani, A. Gomez-Zavaglia, J. Simal-Gandara, Technological strategies ensuring the safe arrival of beneficial microorganisms to the gut: From food processing and storage to their passage through the gastrointestinal tract, Food Research International, 129 (2020) 108852.
[28] K. Feng, R.-m. Huang, R.-q. Wu, Y.-s. Wei, M.-h. Zong, R. J. Linhardt, H. Wu, A novel route for double-layered encapsulation of probiotics with improved viability under adverse conditions, Food chemistry, 310 (2020) 125977.
[29] M. G. Wilkinson, Flow cytometry as a potential method of measuring bacterial viability in probiotic products: a review, (2018).
[30] E. Żymańczyk-Duda, M. Brzezińska-Rodak, M. Klimek-Ochab, M. Duda, A. Zerka, Yeast as a versatile tool in biotechnology, Yeast Ind Appl, 1 (2017) 3-40.
[31] M. Parapouli, A. Vasileiadis, A.-S. Afendra, E. Hatziloukas, Saccharomyces cerevisiae and its industrial applications, AIMS Microbiol, 6 (2020) 1-31.
[32] E. Żymańczyk-Duda, M. Brzezińska-Rodak, M. Klimek-Ochab, M. Duda, A. Zerka, Yeast as a versatile tool in biotechnology, Yeast Ind Appl 1 (2017) 3-40.
[33] M. Parapouli, A. Vasileiadis, A.-S. Afendra, E. Hatziloukas, Saccharomyces cerevisiae and its industrial applications, AIMS Microbiol 6 (2020) 1-31.
[34] S. H. Yang, K. B. Lee, B. Kong, J. H. Kim, H. S. Kim, I. S. Choi, Biomimetic encapsulation of individual cells with silica, Angewandte Chemie International Edition 48 (2009) 9160-9163.
[35] S. H. Yang, K. B. Lee, B. Kong, J. H. Kim, H. S. Kim, I. S. Choi, Biomimetic encapsulation of individual cells with silica, Angewandte Chemie International Edition, 48 (2009) 9160-9163.
[36] S. T. Laughlin, J. M. Baskin, S. L. Amacher, C. R. Bertozzi, In vivo imaging of membrane-associated glycans in developing zebrafish, Science, 320 (2008) 664-667.
[37] J. A. Codelli, J. M. Baskin, N.J. Agard, C. R. Bertozzi, Second-generation difluorinated cyclooctynes for copper-free click chemistry, Journal of the American Chemical Society, 130 (2008) 11486-11493.
[38] E. Saxon, C. R. Bertozzi, Cell surface engineering by a modified Staudinger reaction, Science, 287 (2000) 2007-2010.
[39] L. K. Mahal, K. J. Yarema, C. R. Bertozzi, Engineering chemical reactivity on cell surfaces through oligosaccharide biosynthesis, Science, 276 (1997) 1125-1128.
[40] E. T. Sayed, T. Tsujiguchi, N. Nakagawa, Catalytic activity of baker's yeast in a mediatorless microbial fuel cell, Bioelectrochemistry 86 (2012) 97-101.

What is claimed is:

1. An electrochemical system for encapsulating a probiotic cell with a metallic surface by electrodeposition, the system comprising a two-electrode metal-air battery comprising a cathode, a separator, a carbon cloth, an anode, and an anolyte solution, wherein:
(a) the cathode comprises a Pt/C material;
(b) the separator comprises an ion-conductive membrane;
(c) the anode comprises a carbon cloth modified with a metal nanoshell structure;
(d) the anolyte comprises a probiotic cell.

2. A method for encapsulating a probiotic cell utilizing the system of claim 1, wherein the electrodeposition comprises the step of electrochemically discharging a metal-air battery to form a metallic sheath on the surface of a probiotic cell, wherein the metallic sheath comprises a metal.

3. The method of claim 2, wherein the metal is cobalt.

4. The method of claim 2, wherein the metal is selected from the group consisting of cobalt, zinc, aluminium, magnesium, lithium, iron, zirconium, tungsten, and vanadium.

5. The method of claim 2, wherein the metal air battery comprises cobalt.

6. The method of claim 2, wherein the metal air battery utilizes a metal selected from the group consisting of cobalt, zinc, aluminium, magnesium, lithium, iron, zirconium, tungsten, and vanadium.

7. The method of claim 2, wherein the metallic sheath comprises cobalt.

8. The method of claim 1, wherein the ion-conductive membrane is a polymer or powder resin membrane.

9. The method of claim 1, wherein the polymeric membrane is a Nafion® membrane.

10. The method of claim 2, wherein the metallic sheath has magnetic properties.

11. The method of claim 1, wherein the probiotic cell is a yeast cell.

12. The method of claim 11, wherein the yeast cell is a Saccharomyces cerevisiae cell.

13. The method of claim 1, wherein the probiotic is a microorganism selected from the group consisting of Saccharomyces, Bifidobacterium, Lactobacillus, and Bacillus.

14. The method of claim 1, wherein the system operates at room temperature.

15. The method of claim 2, wherein no current is consumed.

16. The method of claim 2, wherein current is produced.

17. The method of claim 16, wherein the current produced by the battery during discharge has a density of approximately 3 A/m² at 0.3 V.

18. The method of claim 1, wherein the buffer solution of the anolyte has a pH of approximately 7.0.

19. The method of claim 1, wherein the probiotic encapsulated cell is viable and capable of reproducing.

20. The method of claim 10, wherein the magnetic properties of the metallic sheath encapsulating the probiotic cell is utilized in a fuel cell.
